(19) United States
(12) Patent Application Publication
(10) Pub. No.: US 2025/0010033 A1
(43) Pub. Date: Jan. 9, 2025

(54) GUIDE CATHETER WITH BACKUP SUPPORT AND CURVE RETENTION

(71) Applicant: Boston Scientific Medical Device Limited, Galway (IE)

(72) Inventors: Anshul Chabra, Delhi (IN); Sumit Agrawal, Gurgaon (IN); Neeraj Kumar Sharma, New Delhi (IN); Jahnavi Konduru, Gurgaon (IN)

(73) Assignee: Boston Scientific Medical Device Limited, Galway (IE)

(21) Appl. No.: 18/761,687

(22) Filed: Jul. 2, 2024

Related U.S. Application Data
(60) Provisional application No. 63/524,782, filed on Jul. 3, 2023.

(51) Int. Cl. A61M 25/01; A61M 25/00

(52) U.S. Cl. CPC A61M 25/0113; A61M 25/0045; A61M 25/005

(57) ABSTRACT

A guide catheter is adapted for providing improved backup support while accessing an ostium of a patient's left coronary artery. The guide catheter includes an elongate shaft having a proximal shaft portion and a power zone shaft portion. The elongate shaft includes an inner polymeric layer extending through the proximal shaft portion and the power zone shaft portion, and outer polymeric layer, and a braid that is disposed between the inner polymeric layer and the outer polymeric layer and extends through the proximal shaft portion and the power zone shaft portion, the braid having a first PIC count within the power zone shaft portion and a second PIC count within the proximal shaft portion. The elongate shaft is biased into a primary curve disposed distal of the power zone shaft portion and a coplanar secondary curve disposed within the power zone shaft portion.

### CROSS-REFERENCE TO RELATED APPLICATIONS

This application claims the benefit of priority under 35 U.S.C. § 119 of U.S. Provisional Application No. 63/524,782, filed Jul. 3, 2023, the entire disclosure of which is hereby incorporated by reference.

### TECHNICAL FIELD

The present disclosure pertains to medical devices, and methods for manufacturing medical devices. More particularly, the present disclosure pertains to elongated intracorporeal medical devices including a tubular member connected with other structures, and methods for manufacturing and using such devices.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in a guide catheter adapted for providing improved backup support while accessing an ostium of a patient's left coronary artery without deep seating the guide catheter within the ostium. The guide catheter includes an elongate shaft including a proximal shaft portion and a power zone shaft portion. The elongate shaft includes an inner polymeric layer extending through the proximal shaft portion and the power zone shaft portion. The elongate shaft includes an outer polymeric layer including a first outer layer segment formed from a first polymer within the power zone shaft portion and a second outer layer segment formed from a second polymer within the proximal shaft portion. A braid is disposed between the inner polymeric layer and the outer polymeric layer and extends through the proximal shaft portion and the power zone shaft portion, the braid having a first PIC count within the power zone shaft portion and a second PIC count different from the first PIC count within the proximal shaft portion. The elongate shaft is biased into a primary curve disposed distal of the power zone shaft portion. The elongate shaft is biased into a secondary curve disposed within the power zone shaft portion. The secondary curve is coplanar with the primary curve.

Alternatively or additionally, the first outer layer segment may have a first outer diameter and the second outer layer segment may have a second outer diameter less than the first outer diameter.

Alternatively or additionally, the first polymer includes a high stiffness polymer.

Alternatively or additionally, the first polymer includes Vestamid® ME 71.

Alternatively or additionally, the second polymer includes a low stiffness polymer.

Alternatively or additionally, the second polymer includes Pebax® 72D.

Alternatively or additionally, the braid may extend as a single braid through the power zone shaft portion and the proximal shaft portion.

Alternatively or additionally, the braid may be formed from a wire having a 0.0015 inch by 0.005 inch cross-sectional profile.

Alternatively or additionally, the braid may have a 65 PIC count within the proximal shaft portion and the braid may have a 60 PIC count within the power zone shaft portion.

Alternatively or additionally, the primary curve may include a bend in a range of 75° to 100°.

Alternatively or additionally, the secondary curve may include a bend in a range of 150° to 170°.

Another example may be found in a guide catheter adapted for providing improved backup support while accessing an ostium of a patient's left coronary artery without deep seating the guide catheter within the ostium. The guide catheter includes an elongate shaft extending from a distal end to a proximal end, the elongate shaft including a proximal shaft portion and a power zone shaft portion. The elongate shaft includes an inner polymeric layer extending from the distal end to the proximal end and an outer polymeric layer, the outer polymeric layer formed from a first polymer within the power zone shaft portion and a second polymer within the proximal shaft portion. A braid is disposed between the inner polymeric layer and the outer polymeric layer and extends from the distal end to the proximal end, the braid having a first PIC count within the power zone shaft portion and a second PIC count different from the first PIC count within the proximal shaft portion. The elongate shaft is biased into a first curve having a first bend in a range of 75° to 100° and a second curve having a second bend in a range of 150° to 170°. The second curve is coplanar with the first curve.

Alternatively or additionally, the first curve may be disposed distal of the power zone shaft portion.

Alternatively or additionally, the second curve may be disposed within the power zone shaft portion.

Alternatively or additionally, the elongate shaft may have a first outer diameter within the power zone shaft portion and a second outer diameter within the proximal shaft portion.

Alternatively or additionally, the first outer diameter may be up to 5 percent greater than the second outer diameter.

Alternatively or additionally, the first outer diameter may be 1 to 2 percent greater than the second outer diameter.

Another example may be found in a guide catheter adapted for providing improved backup support while accessing an ostium of a patient's left coronary artery without deep seating the guide catheter within the ostium. The guide catheter includes a 6 French elongate shaft including a proximal shaft portion and a power zone shaft portion. The elongate shaft includes an inner polymeric layer extending through the proximal shaft portion and the power zone shaft portion and an outer polymeric layer. The outer polymeric layer includes a first outer layer segment formed from a first polymer within the power zone shaft portion and a second outer layer segment formed from a second polymer within the proximal shaft portion. A braid is disposed between the inner polymeric layer and the outer polymeric layer and extending through the proximal shaft portion and the power zone shaft portion, the braid having a 60 PIC count within the power zone shaft portion and a 65 PIC count within the proximal shaft portion. The 6 French elongate shaft is biased into a primary curve disposed distal of the power zone shaft portion and a secondary curve disposed within the power zone shaft portion. The secondary curve is coplanar with the primary curve.

Alternatively or additionally, the first outer layer segment may include Vestamid® ME 71 that includes 20 percent bismuth subcarbonate and the first outer layer segment may have an outer diameter of 0.0825 inches.

Alternatively or additionally, the second outer layer segment may include Pebax® 72D that includes 20 percent bismuth subcarbonate and the second outer layer segment may have an outer diameter of 0.0840 inches.

The above summary of some embodiments is not intended to describe each disclosed embodiment or every implementation of the present disclosure. The Figures and Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic view of an illustrative guide catheter;

FIG. 1A is an enlarged view of the illustrative guide catheter of FIG. 1, annotating geometry of the guide catheter;

FIG. 2 is a cross-sectional view taken along the line 2-2 of FIG. 1;

FIG. 3 is a cross-sectional view taken along the line 3-3 of FIG. 1;

FIG. 4 is a cross-sectional view taken along the line 4-4 of FIG. 1;

FIG. 5 is a schematic view of the illustrative guide catheter of FIG. 1, shown within the aortic arch accessing the left coronary artery ostium;

FIG. 6A is a graph of experimental data showing backup support data for different outer layer polymers at ten minutes;

FIG. 6B is a graph of experimental data showing backup support data for different outer layer polymers at forty minutes;

FIG. 7 is a graph of experimental data showing curve retention data for different outer layer polymers at forty minutes;

FIG. 8A is a graph of experimental data showing backup support data for various braid designs at ten minutes;

FIG. 8B is a graph of experimental data showing backup support data for various braid designs at forty minutes;

FIG. 9 is a graph of experimental data showing torque to kink data for various braid designs at forty minutes;

FIG. 10 is a graph of experimental data showing curve retention for various braid designs at forty minutes; and

FIG. 11A is a graph of experimental data showing backup support data for various outer material thicknesses at ten minutes; and

FIG. 11B is a graph of experimental data showing backup support data for various outer material thicknesses at forty minutes.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

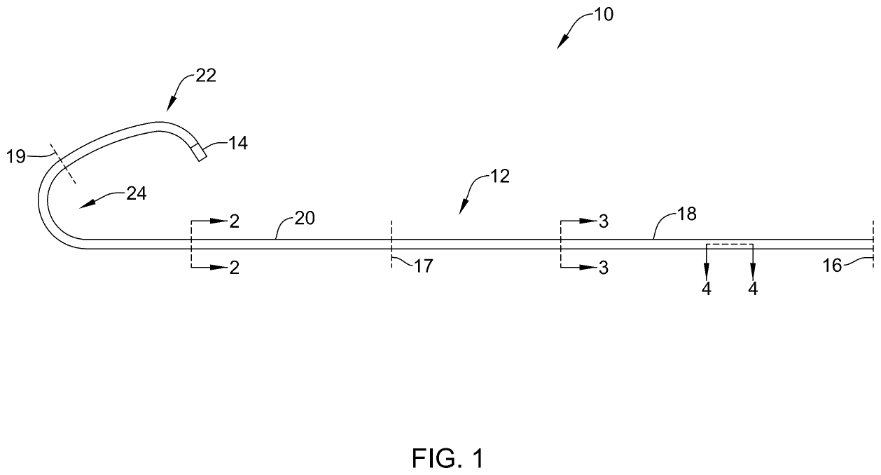

FIG. 1 is a schematic view of an illustrative guide catheter 10. The illustrative guide catheter 10 may be considered as being adapted for providing improved backup support while accessing an ostium of a patient's left coronary artery from a position within the ascending aorta without resorting to deep seating the guide catheter 10 within the ostium. The guide catheter 10 includes an elongate shaft 12 that extends from a distal end 14 to a proximal end 16. The distal end 14 is adapted to access a left coronary artery ostium. While not shown, the proximal end 16 may include additional structure such as a hub, for example. The elongate shaft 12 has several regions, including a proximal shaft portion 18 and a power zone shaft portion 20. The proximal shaft portion 18 extends form the proximal end 16 to a transition point 17. The power zone shaft portion 20 extends from the transition point 17 to a transition point 19. As will be discussed, one or more characteristics of the elongate shaft 12 may change at the transition point 17 and the transition point 19. While referred to as transition points, the transition points 17 and 19 may in some instances be considered as transition regions, in which one or more characteristics of the elongate shaft 12 change over a length, rather than changing abruptly.

The elongate shaft 12 may include a polymeric inner layer or liner that is formed of a lubricious material such as but not limited to polytetrafluoroethylene and that extends through the elongate shaft 12 from the distal end 14 to the proximal end 16. The elongate shaft 12 may include an outer polymeric layer that extends from the distal end 14 to the proximal end 16, but may include a different polymer or polymers within the proximal shaft portion 18 relative to the polymer or polymers utilized within the power zone shaft portion 20. In some instances, the outer polymeric layer may have a first outer diameter within the power zone shaft portion 20 and a second outer diameter within the proximal shaft portion 18. A reinforcing braid may extend through the proximal shaft portion 18 and the power zone shaft portion 20, but may have a first PIC (per inch crossings) value within the power zone shaft portion 20 and a second PIC value within the proximal shaft portion 18, even though the elongate shaft 12 includes a singular braid structure that extends through both the proximal shaft portion 18 and the power zone shaft portion 20.

As seen, the guide catheter 10 may be adapted to include a primary curve 22 that is located distal of the power zone shaft portion 20. In some instances, the guide catheter 10 may be adapted to include a secondary curve 24 that is located within the power zone shaft portion 20. In some instances, the guide catheter 10 may be adapted to bias the elongate shaft 12 into including the primary curve 22 and the secondary curve 24. While the elongate shaft 12 may be deflected or deformed away from its biased configuration, the elongate shaft 12 may try to regain its biased configuration, including the primary curve 22 and the secondary curve 24. In some instances, the proximal shaft portion 18 may be adapted to provide appropriate characteristics for advancing the guide catheter 10 through the vasculature while the power zone shaft portion 16 may be adapted to provide appropriate characteristics for aligning the guide catheter 10 with the appropriate ostium.

FIG. 1A is an enlarged view of a distal portion of the guide catheter 10, annotated to call out geometric features of the elongate shaft 12. As can be seen, the primary curve 22 may be considered as forming an angle α₁ as measured between a distal region 26 of the elongate shaft 12 distal of the primary curve 22 and an intermediate region 28 of the elongate shaft 12 proximal of the primary curve 22. In some instances, the primary curve 22 may be biased to form an angle α₁ that is in a range of 75° to 100°. In some instances, the primary curve 22 may be biased to form an angle α₁ that is about 90°. The secondary curve 24 may be considered as forming an angle α₂ as measured between the intermediate region 28 of the elongate shaft 12 distal of the power zone shaft portion 20 and the power zone shaft portion 20 proximal of the secondary curve 24. In some instances, the secondary curve 24 may be biased to form an angle α₂ that is in a range of 150° to 170°. In some instances, secondary curve 24 may be biased to form an angle α₂ that is about 160°. In some instances, the primary curve 22 and the secondary curve 24 may be considered as being coplanar, or lying within the same plane. As an example, if the elongate shaft 12 is considered as extending along a plane defined by a surface along which the elongate shaft 12 is resting, the entire elongate shaft 12 is within that same plane. Neither the primary curve 22 nor the secondary curve 24 are considered as being biased into a configuration in which the primary curve 22 or the secondary curve 24 curve below that surface, or curve above that surface.

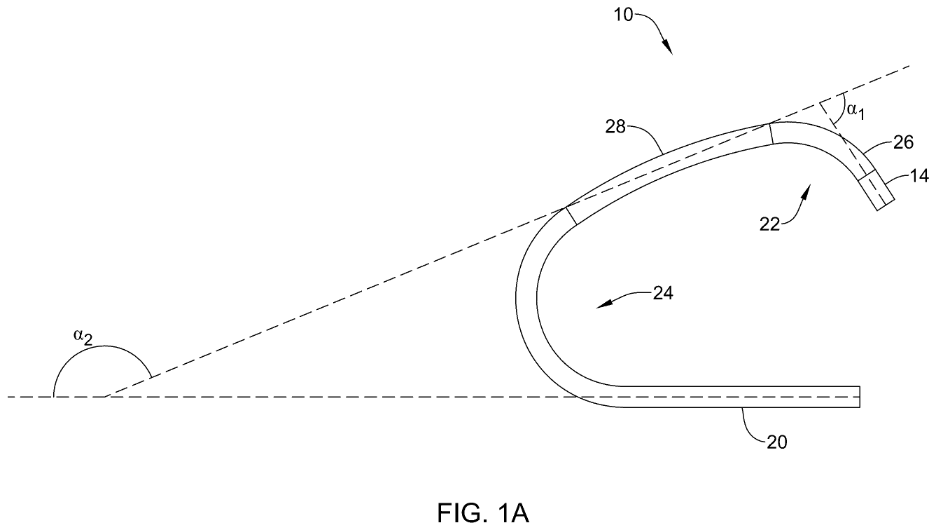

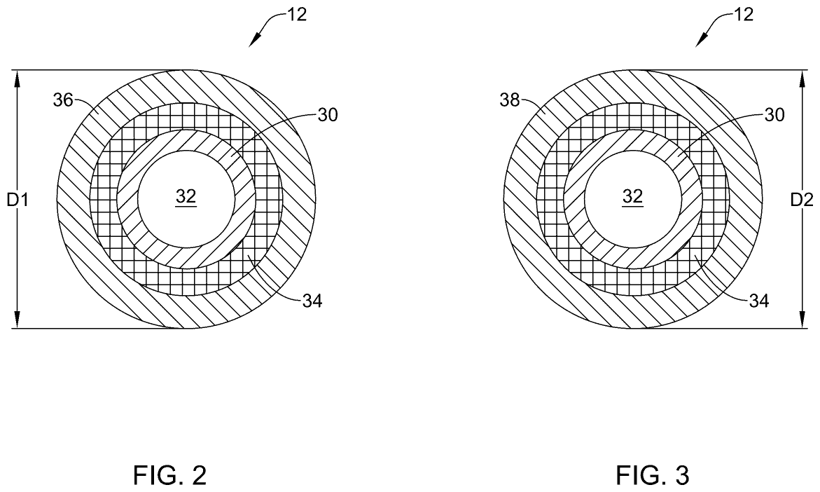

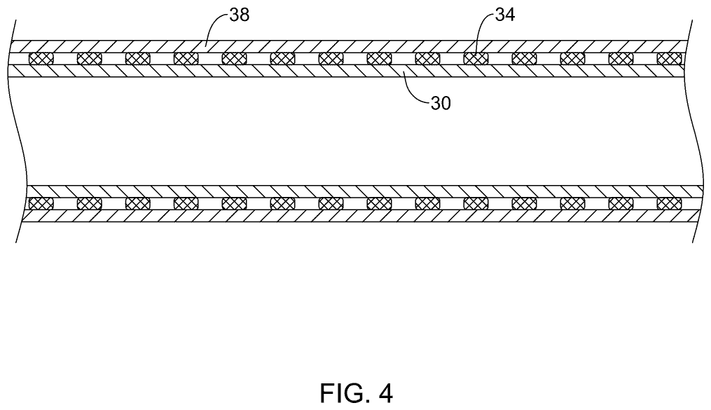

FIG. 2 is a cross-sectional view taken along the line 2-2, within the power zone shaft portion 20 while FIG. 3 is a cross-sectional view taken along the line 2-2, within the proximal shaft portion 18. FIG. 4 is a longitudinal cross-sectional view taken along the line 4-4, also within the proximal shaft portion 18. The elongate shaft 12 includes an inner polymeric layer 30 defining a lumen 32 extending within the inner polymeric layer 30. The inner polymeric layer 30 extends from the distal end 14 of the elongate shaft 12 to the proximal end 16 of the elongate shaft 12. The inner polymeric layer 30 may be formed of any suitable polymer. In some instances, the inner polymeric layer 30 may be formed of a lubricious polymer such as a fluoropolymer. Examples of suitable fluoropolymers include polyvinylfluoride (PVF), polyvinylidene fluoride (PVDF), polytetrafluoroethylene (PTFE), perfluoroalkoxy (PFA) polymer, fluorinated ethylene-propylene (FEP) and polyethylenetetrafluoroethylene (ETFE), among others. In some instances, the inner polymeric layer 30 may have an inner diameter in a range of 0.056 inches to 0.095 inches, for example, and an outer diameter in a range of 0.058 inches to 0.097 inches. In some instances, the inner polymeric layer 30 may have a wall thickness of 0.001 inches. It will be appreciated that these ranges are applicable for guide catheters having a size of 5 French to 8 French, for example.

The elongate shaft 12 includes a reinforcing braid 34 that extends through both the proximal shaft portion 18 and the power zone shaft portion 20. In some instances, the reinforcing braid 34 also extends through the intermediate region 28 of the elongate shaft 12 and even through the distal region 26 of the elongate shaft 12. In some instances, the reinforcing braid 34 does not extend distally beyond the power zone shaft portion 20. A unitary braid 34 extends through both the proximal shaft portion 18 and the power zone shaft portion 20. In some instances, the braid 34 may have a first PIC count within the power zone shaft portion 20 and a second PIC count different from the first PIC count within the proximal shaft portion 18. PIC count refers to per inch crosses, which is a measure of how tightly formed the braid 34 is. The first PIC count within the power zone shaft portion 20 may be 50-80, or 60-70, or 65. The second PCI count within the proximal shaft portion 18 may be 40-75, or 55-65, or 60. In some instances, the braid 34 may have a higher PIC count within the proximal shaft portion 18 and a relatively lower PIC count within the power zone shaft portion 20. As an example, the braid 34 may have a PIC count of 65 within the proximal shaft portion 18 and a PIC count of 60 within the power zone shaft portion 20. In some instances, the braid 34 may be a 1×1 braid, or a 2×2 braid, or a 3×3 braid, or a 4×4 braid, which refers to how many individual braid wires extend helically in each direction in forming the braid 34.

In some instances, the reinforcing braid 34 may include one or more wires that have a substantially circular cross-sectional shape. In some of these and in other instances, the reinforcing braid 34 may include one or more wires that have a non-circular cross-sectional shape. This may include a polygonal shape such as rectangular shape. For example, the reinforcing braid 34 may include wires having a width greater than the thickness (e.g., about 0.0015 inches by 0.0005 inches), as seen for example in FIG. 4. This is not intended to be limiting. Other dimensions and shapes are contemplated. In some instances, the braid 34 may be formed from a flat wire such as a ribbon wire having an 0.0015 inches by 0.005 inches cross-sectional profile. In some instances, the braid 34 may be formed of materials such as stainless steel including SST 304 or tungsten, although other materials are contemplated.

The elongate shaft 12 includes an outer polymeric layer that varies between the proximal shaft portion 18 and the power zone shaft portion 20. In some instances, the outer polymeric layer may include a first outer layer segment 36 within the power zone shaft portion 20 and a second outer layer segment 38 within the proximal shaft portion 18. In some instances, the first outer layer segment 36 may be formed of a first polymer and the second outer layer segment 38 may be formed of a second polymer. In some instances, the first outer layer segment 36 may be formed of a low stiffness polyether block amide material while the second outer layer segment 38 may be formed of a high stiffness polyether block amide material. In an example, the first outer layer segment 36 may be formed of Pebax® 72D while the second outer layer segment 38 may be formed of Vestamid® ME 71.

In some instances, the first outer layer segment 36 may be considered as having an outer diameter as indicated by D1 in FIG. 3 and the second outer layer segment 38 may be considered as having an outer diameter as indicated by D2 in FIG. 4. In some instances, the diameter D1 may be larger than the diameter D2. As an example, D1 may be as much as 5 percent larger than D2. As another example, D1 may be 1 to 2 percent larger than D2. In an example in which the guide catheter 10 is sized as a 5 French catheter, D1 may be equal to 0.071 inches while D2 may be equal to 0.069 inches. In an example in which the guide catheter 10 is sized as a 6 French catheter, D1 may be equal to 0.0840 inches while D2 may be equal to 0.0825 inches. In an example in which the guide catheter 10 is sized as a 7 French catheter, D1 may be equal to 0.095 inches while D2 may be equal to 0.0935 inches. In an example in which the guide catheter 10 is sized as a 8 French catheter, D1 may be equal to 0.113 inches while D2 may be equal to 0.110 inches. Similar dimensions may be provided for other sizes of guide catheters.

In some instances, the outer polymeric layer may include a radiopaque material in order to cause the guide catheter 10 to more clearly show up during fluoroscopy and other imaging processes. As an example, a radiopaque material may be dispersed within the polymer forming the first outer layer segment 36 within the power zone shaft portion 20. A radiopaque material may be dispersed within the polymer forming the second outer layer segment 38 within the proximal shaft portion 18. In some instances, bismuth subcarbonate (Bi₂O₂CO₃) or barium sulfate (BaSO₄) may be used as the radiopaque material. Other radiopaque materials are also contemplated.

In some instances, the polymer forming the first outer layer segment 36 may include 20 weight percent bismuth subcarbonate and the polymer forming the second outer layer segment 38 may include 20 weight percent bismuth subcarbonate. While this is given as a particular example, it will be appreciated that in some instances, the polymer may include a different weight percent of bismuth subcarbonate. In some instances, the polymer may include a different radiopaque material. In some instances, inclusion of radiopaque fillers such as barium sulfate and bismuth subcarbonate can change the overall properties of the polymer used to form the first outer layer segment 36. In some instances, the relative amounts of radiopaque filler may be varied, depending on location. As an example, the first outer layer segment 36 may be formed of Pebax® 72D plus twenty percent bismuth subcarbonate while the second outer layer segment 38 may be formed of Vestamid® ME 71 and ten percent barium sulfate.

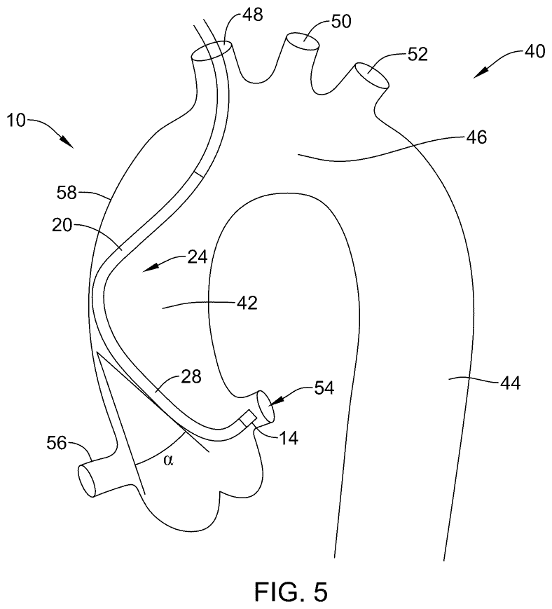

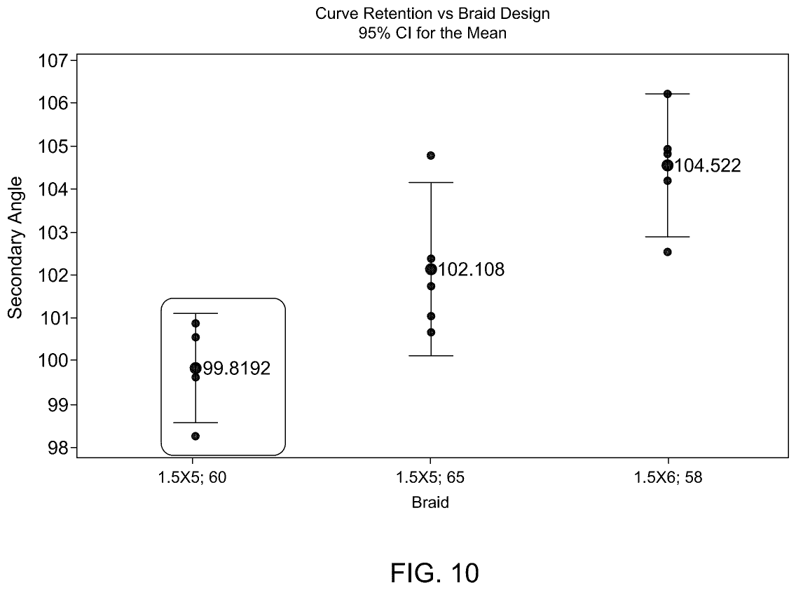

FIG. 5 shows the guide catheter 10 disposed within the vasculature. In particular, FIG. 10 is a schematic view of an aorta 40 including an ascending aorta 42, a descending aorta 44 and an aortic arch 46 disposed between the ascending aorta 42 and the descending aorta 44. The aortic arch 46 is fluidly coupled with a brachiocephalic artery 48, which bifurcates into the right subclavian artery and the right common carotid artery. The aortic arch 46 is fluidly coupled with a left common carotid artery 50 and a left subclavian artery 52. As shown, the guide catheter 10 has been advanced through the brachiocephalic artery 48 and into the ascending aorta 42. The distal end 14 of the guide catheter 10 is positioned within an ostium 54 of the left coronary artery. It can be seen that the power zone shaft portion 20 contacts a wall 58 of the ascending aorta 42. The secondary curve 24 helps to position the distal end 14 of the guide catheter 10 proximate the ostium 54. The guide catheter 10 forms an angle α between the wall 58 of the ascending aorta 42 and the intermediate region 28 of the elongate shaft 12. In some instances, the angle α may be in a range of 30 degrees to 45 degrees, although if a physician deep seats the distal end 14 of the guide catheter 10, the angle α may be in a range of 45 degrees to 60 degrees.

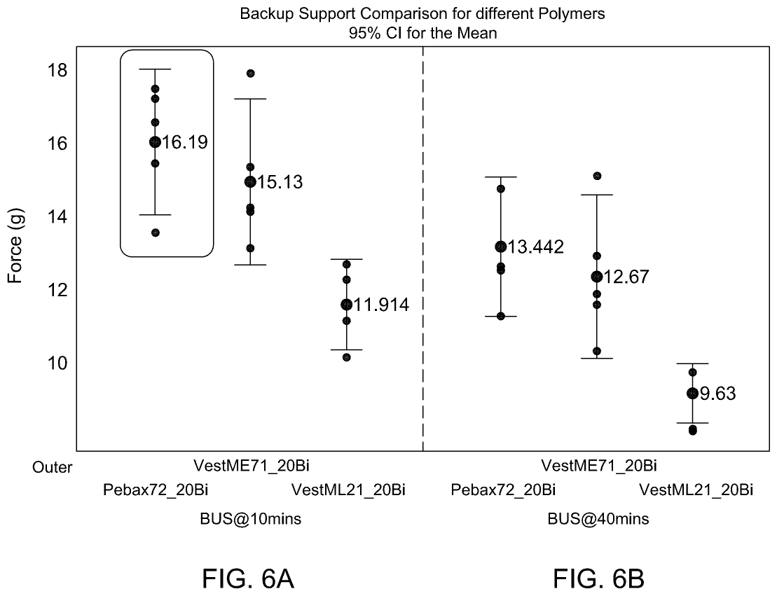

FIG. 6A is a graph of experimental data showing backup support data for different outer layer polymers at ten minutes. The backup support data was collected using a water bath at 37° C. in order to simulate the thermal effects that being in the body would have on the polymers used in the tested guide catheter. In FIG. 6A, Pebax® 72D with 20 weight percent bismuth subcarbonate was tested as an outer material against several Vestamid® polymers. As can be seen, the Pebax® 72D with 20 weight percent bismuth subcarbonate exhibited a higher force when compared with the Vestamid® polymers, indicating better backup support. FIG. 6B is a graph of experimental data showing backup support data for the same polymers after forty minutes in the water bath. As can be seen, the force values have declined for each of the tested polymers, as a result of the polymers undergoing thermal softening, but the Pebax® 72D with 20 weight percent bismuth subcarbonate still demonstrate a greater force, indicating better backup support, than the tested Vestamid® polymers.

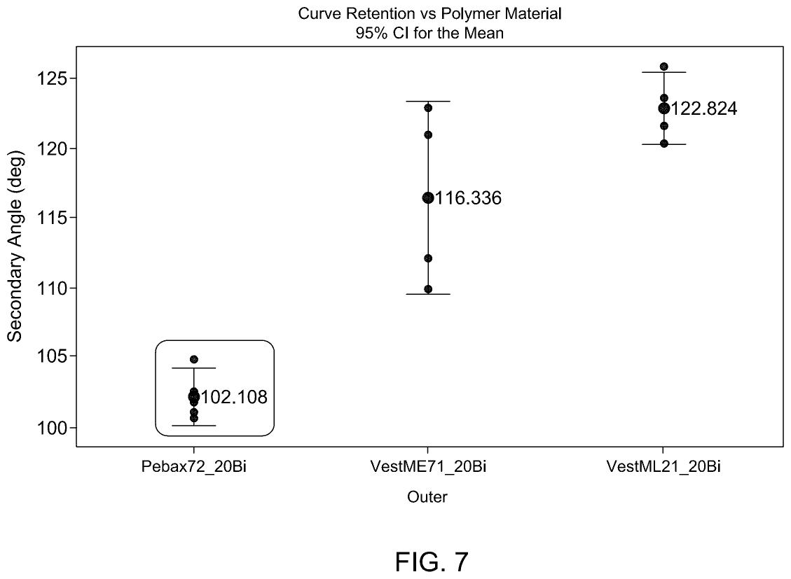

FIG. 7 is a graph of experimental data showing curve retention data for different outer layer polymers at forty minutes. In this, a lower number is better. Pebax® 72D with 20 weight percent bismuth subcarbonate was tested as an outer material against several Vestamid® polymers. As can be seen, the Pebax® 72D with 20 weight percent bismuth subcarbonate resulted in a lower secondary angle relative to the Vestamid® polymers. This shows that the Pebax® 72D with 20 weight percent bismuth subcarbonate tested better than the Vestamid® polymers, and thus is an optimal material to be used for the outer polymer layer of the power zone shaft portion 20.

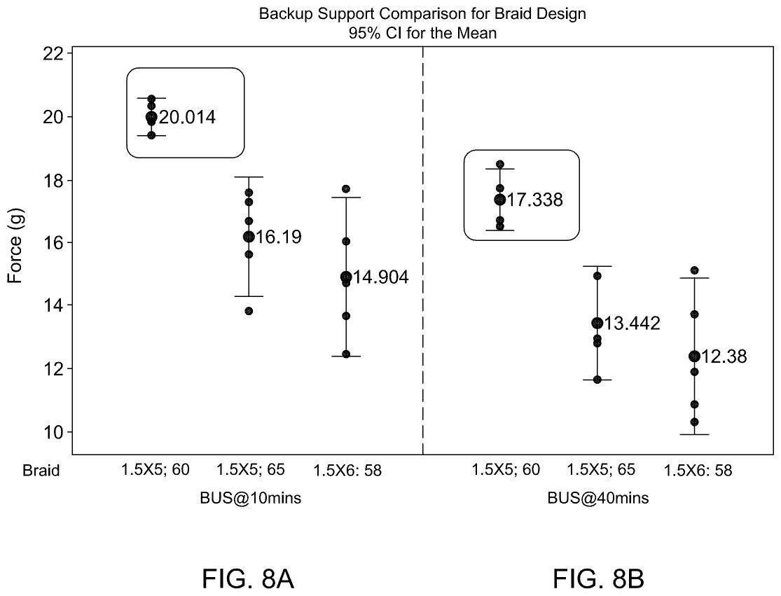

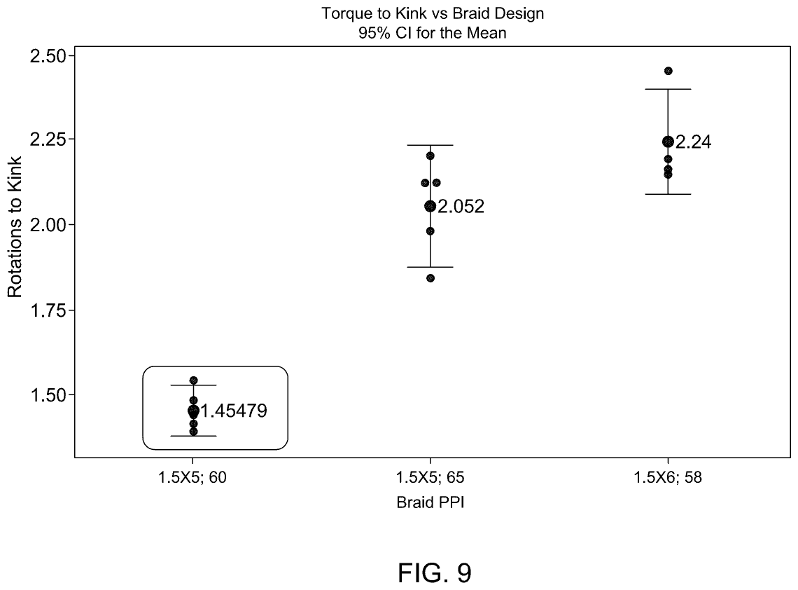

FIG. 8A is a graph of experimental data showing backup support data for various braid designs at ten minutes. A 1.5×5 (0.0015 inches by 0.005 inches) braid was tested at a 60 PIC and a 65 PIC, as well as a 1.5×6 (0.0015 inches by 0.006 inches) tested at a 58 PIC. Each braid was tested with Pebax® 72D with 20 weight percent bismuth subcarbonate as an outer material. FIG. 8B is a graph of experimental data showing backup support data for the same braid designs at forty minutes. As can be seen, the 1.5×5 braid with a PIC count of 60 demonstrated better backup support at ten minutes and at forty minutes. FIG. 9 is a graph of experimental data showing torque to kink data for various braid designs. As can be seen, the 1.5×5 braid with a 60 PIC exhibited a lower torque to kink. However, because backup support performance was prioritized, the 1.5×5 wire was selected.

FIG. 10 is a graph of experimental data showing curve retention for the same braid designs as those shown in FIGS. 8A and 8B, albeit after forty minutes in the water bath. As can be seen, the 1.5×5 braid at 60 PIC exhibited a lower angle and thus a better curve retention in comparison with the other tested braid designs.

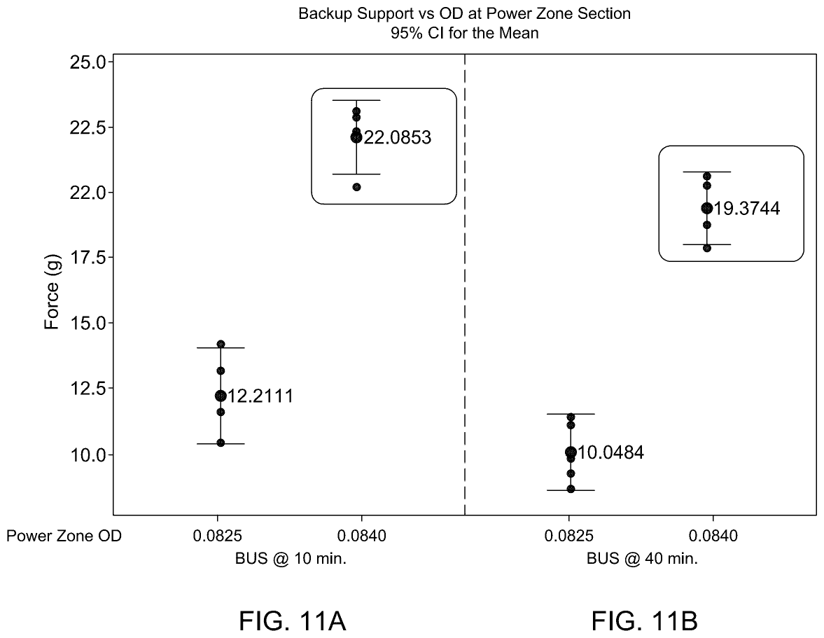

FIG. 11A is a graph of experimental data showing backup support data for various outer material thicknesses at ten minutes while FIG. 11B shows the same data at forty minutes. As can be seen, the sample with a 0.0840 inch diameter performed better at both ten minutes and at forty minutes with respect to the 0.0825 inch diameter

The materials that can be used for the various components of the medical devices described herein may include those commonly associated with medical devices. The medical devices described herein, as well as individual components thereof, be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear clastic and/or non-super-elastic nitinol does not display a substantial “super-clastic plateau” or “flag region” in its stress/strain curve like super-elastic nitinol does. Instead, in the linear-clastic and/or non-super-elastic nitinol, as recoverable strain increases, the stress continues to increase in a substantially linear, or a somewhat, but not necessarily entirely linear relationship until plastic deformation begins or at least in a relationship that is more linear that the super-elastic plateau and/or flag region that may be seen with super-elastic nitinol. Thus, for the purposes of this disclosure linear-clastic and/or non-super-clastic nitinol may also be termed “substantially” linear-clastic and/or non-super-clastic nitinol.

In some cases, linear elastic and/or non-super-elastic nitinol may also be distinguishable from super-elastic nitinol in that linear-elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that also can be distinguished based on its composition), which may accept only about 0.2 to 0.44 percent strain before plastically deforming.

In some embodiments, the linear-elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (C) to about 120° C. in the linear-clastic and/or non-super-elastic nickel-titanium alloy. The mechanical bending properties of such material may therefore be generally inert to the effect of temperature over this very broad range of temperature. In some embodiments, the mechanical bending properties of the linear-elastic and/or non-super-elastic nickel-titanium alloy at ambient or room temperature are substantially the same as the mechanical properties at body temperature, for example, in that they do not display a super-clastic plateau and/or flag region. In other words, across a broad temperature range, the linear-clastic and/or non-super-elastic nickel-titanium alloy maintains its linear-elastic and/or non-super-elastic characteristics and/or properties.

In some embodiments, the linear-elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a super-elastic alloy, for example a super-elastic nitinol can be used to achieve desired properties.

In at least some embodiments, portions or all of the medical devices described herein may also be doped with, made of, or otherwise include a radiopaque material. Radiopaque materials are understood to be materials capable of producing a relatively bright image on a fluoroscopy screen or another imaging technique during a medical procedure. This relatively bright image aids in determining a location of a medical device that includes a radiopaque material. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into various medical devices to achieve the same result.

The medical devices described herein, as well as portions and components thereof, may be made of the same material along its length, or in some embodiments, can include portions or sections made of different materials. In some embodiments, materials may be chosen to impart varying flexibility and stiffness characteristics to different portions. For example, different portions of a component, such as a proximal section and a distal section, may be formed of different materials, for example, materials having different moduli of elasticity, resulting in a difference in flexibility. In some embodiments, the material used to construct a proximal section may be relatively stiff for pushability and torqueability, and the material used to construct a distal section may be relatively flexible by comparison for better lateral trackability and steerability. For example, a proximal section may be formed of straightened 304v stainless steel wire or ribbon and a distal section may be formed of a straightened super elastic or linear elastic alloy, for example a nickel-titanium alloy wire or ribbon.

In embodiments where different portions of the medical devices described herein are made of different materials, the different portions can be connected using a suitable connecting technique and/or with a connector. For example, the different portions may be connected using welding (including laser welding), soldering, brazing, adhesive, or the like, or combinations thereof. These techniques can be utilized regardless of whether or not a connector is utilized. An example of a connector is a structure such as a hypotube or a coiled wire which has an inside diameter sized appropriately to receive and connect to the ends of the proximal portion and the distal portion.

A sheath or covering (not shown) may be disposed over portions or all of the medical devices described herein. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex® high-density polyethylene, Marlex® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the medical devices described herein may be sandblasted, beadblasted, sodium bicarbonate-blasted, electropolished, etc. In these as well as in some other embodiments, a coating, for example a lubricious, a hydrophilic, a protective, or other type of coating may be applied over portions or all of the medical devices described herein. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve stecrability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A guide catheter adapted for providing improved backup support while accessing an ostium of a patient's left coronary artery without deep seating the guide catheter within the ostium, the guide catheter comprising:
an elongate shaft including a proximal shaft portion and a power zone shaft portion, the elongate shaft including:
an inner polymeric layer extending through the proximal shaft portion and the power zone shaft portion;
an outer polymeric layer including:
a first outer layer segment formed from a first polymer within the power zone shaft portion; and
a second outer layer segment formed from a second polymer within the proximal shaft portion; and
a braid disposed between the inner polymeric layer and the outer polymeric layer and extending through the proximal shaft portion and the power zone shaft portion, the braid having a first PIC count within the power zone shaft portion and a second PIC count different from the first PIC count within the proximal shaft portion;
wherein:
the elongate shaft is biased into a primary curve disposed distal of the power zone shaft portion;
the elongate shaft is biased into a secondary curve disposed within the power zone shaft portion, and
the secondary curve is coplanar with the primary curve.

2. The guide catheter of claim 1, wherein the first outer layer segment has a first outer diameter and the second outer layer segment has a second outer diameter less than the first outer diameter.

3. The guide catheter of claim 1, wherein the first polymer comprises a high stiffness polymer.

4. The guide catheter of claim 1, wherein the first polymer comprises Vestamid® ME 71.

5. The guide catheter of claim 1, wherein the second polymer comprises a low stiffness polymer.

6. The guide catheter of claim 1, wherein the second polymer comprises Pebax® 72D.

7. The guide catheter of claim 1, wherein the braid extends as a single braid through the power zone shaft portion and the proximal shaft portion.

8. The guide catheter of claim 1, wherein the braid is formed from a wire having a 0.0015 inch by 0.005 inch cross-sectional profile.

9. The guide catheter of claim 1, wherein the braid has a 65 PIC count within the proximal shaft portion and the braid has a 60 PIC count within the power zone shaft portion.

10. The guide catheter of claim 1, wherein the primary curve includes a bend in a range of 75° to 100°.

11. The guide catheter of claim 1, wherein the secondary curve includes a bend in a range of 150° to 170°.

12. A guide catheter adapted for providing improved backup support while accessing an ostium of a patient's left coronary artery without deep seating the guide catheter within the ostium, the guide catheter comprising:
an elongate shaft extending from a distal end to a proximal end, the elongate shaft including a proximal shaft portion and a power zone shaft portion, the elongate shaft including:
an inner polymeric layer extending from the distal end to the proximal end;
an outer polymeric layer, the outer polymeric layer formed from a first polymer within the power zone shaft portion and a second polymer within the proximal shaft portion; and
a braid disposed between the inner polymeric layer and the outer polymeric layer and extending from the distal end to the proximal end, the braid having a first PIC count within the power zone shaft portion and a second PIC count different from the first PIC count within the proximal shaft portion;
wherein:
the elongate shaft is biased into a first curve having a first bend in a range of 75° to 100°;
the elongate shaft is biased into a second curve having a second bend in a range of 150° to 170°; and
the second curve is coplanar with the first curve.

13. The guide catheter of claim 12, wherein the first curve is disposed distal of the power zone shaft portion.

14. The guide catheter of claim 12, wherein the second curve is disposed within the power zone shaft portion.

15. The guide catheter of claim 12, wherein the elongate shaft has a first outer diameter within the power zone shaft portion and a second outer diameter within the proximal shaft portion.

16. The guide catheter of claim 15, wherein the first outer diameter is up to 5 percent greater than the second outer diameter.

17. The guide catheter of claim 15, wherein the first outer diameter is 1 to 2 percent greater than the second outer diameter.

18. A guide catheter adapted for providing improved backup support while accessing an ostium of a patient's left coronary artery without deep seating the guide catheter within the ostium, the guide catheter comprising:
a 6 French elongate shaft including a proximal shaft portion and a power zone shaft portion, the elongate shaft including:
an inner polymeric layer extending through the proximal shaft portion and the power zone shaft portion;
an outer polymeric layer including:
a first outer layer segment formed from a first polymer within the power zone shaft portion; and
a second outer layer segment formed from a second polymer within the proximal shaft portion; and
a braid disposed between the inner polymeric layer and the outer polymeric layer and extending through the proximal shaft portion and the power zone shaft portion, the braid having a 60 PIC count within the power zone shaft portion and a 65 PIC count within the proximal shaft portion;
wherein:
the 6 French elongate shaft is biased into a primary curve disposed distal of the power zone shaft portion;
the 6 French elongate shaft is biased into a secondary curve disposed within the power zone shaft portion, and
the secondary curve is coplanar with the primary curve.

19. The guide catheter of claim 18, wherein the first outer layer segment comprises Vestamid® ME 71 that includes 20 percent bismuth subcarbonate and the first outer layer segment has an outer diameter of 0.0825 inches.

20. The guide catheter of claim 18, wherein the second outer layer segment comprises Pebax® 72D that includes 20 percent bismuth subcarbonate and the second outer layer segment has an outer diameter of 0.0840 inches.
